(19) United States
(12) Patent Application Publication
(10) Pub. No.: US 2025/0352664 A1
(43) Pub. Date: Nov. 20, 2025

(54) METH0DS OF ADMINISTERING LIPID NANOPARTICLES INCLUDING POLY(ETHYLOXAZOLINE)-LIPID CONJUGATES WITHOUT RAISING IMMUNE RESPONSE TO POLYMER

(71) Applicant: Serina Therapeutics (AL), Inc., Hunstville, AL (US)

(72) Inventors: Randall Moreadith, Huntsville, AL (US); Kelley Smith, Madison, AL (US); Wookhyun Kim, Madison, AL (US)

(73) Assignee: Serina Therapeutics (AL), Inc., Huntsville, AL (US)

(21) Appl. No.: 19/279,845

(22) Filed: Jul. 24, 2025

Related U.S. Application Data
(63) Continuation-in-part of application No. 18/387,528, filed on Nov. 7, 2023.
Application No. 18/387,528 is a division of application No. 17/665,190, filed on Feb. 4, 2022, now Pat. No. 12,233,132.
(60) Provisional application No. 63/676,530, filed on Jul. 29, 2024.

(51) Int. Cl. A61K 47/69; A61K 39/00

(52) U.S. Cl. CPC A61K 47/6935; A61K 39/00

(57) ABSTRACT

Methods of administering lipid nanoparticle compositions including poly(ethyloxazoline)-lipid conjugates (PEOZ-lipid LNPs) without triggering a PEOZ-associated immune response. In some aspects, the PEOZ-lipid LNPs trigger a protective response based on the encapsulated payload but do not trigger an IgM or IgG response. also provides an absent or. In other aspects, the PEOZ-lipid LNPs trigger a protective response based on the encapsulated payload but trigger an IgM and IgG response that is markedly reduced (as compared to a comparable PEG-lipid currently used in LNP vaccine delivery systems). PEOZ-DMA LNPs incorporating payloads such as oligonucleotides payloads mRNA, DNA, and siRNA for delivery into living cells is also contemplated.

### CROSS-REFERENCE TO RELATED APPLICATIONS

This application claims priority to U.S. Provisional Patent Application No. 63/676,530, filed Jul. 29, 2024, and is also a continuation-in-part of U.S. patent application Ser. No. 18/387,528, filed Nov. 7, 2023, now pending, which is a divisional application of U.S. patent application Ser. No. 17/665,190, filed Feb. 4, 2022, now U.S. Pat. No. 12,233,132, the entire disclosures of which are incorporated by reference herein.

### FIELD OF THE DISCLOSURE

The present disclosure relates to methods of administering lipid nanoparticle compositions including poly (ethyloxazoline)-lipid conjugates (PEOZ-lipid LNPs) without triggering a PEOZ-associated immune response. In some aspects, the PEOZ-lipid LNPs trigger a protective response based on the encapsulated payload but do not trigger an IgM or IgG response. In other aspects, the PEOZ-lipid LNPs trigger a protective response based on the encapsulated payload but trigger an IgM or IgG response that is less than about 0.1 percent higher than the IgM or IgG prior to administration of the PEOZ-lipid LNP (and significantly less than the PEG-lipid LNP counterpart). PEOZ-DMA LNPs incorporating payloads such as oligonucleotides payloads mRNA, DNA, and siRNA for delivery into living cells is also contemplated.

### BACKGROUND

Since lipid nanoparticles (LNPs) are generally considered to be biocompatible nanocarriers with an acceptable safety profile and capacity to carry oligonucleotide payloads, efforts to develop better vaccine delivery systems have resulted in encapsulation of oligonucleotide payloads into (LNPs). For example, approved mRNA vaccines for COVID-19 from Pfizer/BioNTech and Moderna both use antigenic mRNA encoding a nucleoside-modified prefusion form of the spike antigen (S-2P) packaged in LNPs for delivery. Such LNPs are composed of an ionizable lipid (which complexes with the oligonucleotide), cholesterol (to provide flexibility), a helper lipid (to provide structural integrity), and a lipid that includes a polyethylene glycol (PEG) moiety (to stabilize the lipid nanoparticles and prevent fusion with other nanoparticles).

However, it is acknowledged that treating patients with PEGylated components, including PEG-lipids, can lead to the formation of antibodies that specifically recognize and bind to PEG (i.e., anti-PEG antibodies). Indeed, although free PEG is generally poorly immunogenic, it may induce anti-PEG antibodies (IgG and IgM) upon conjugation with other materials such as proteins and nanocarriers. A 2022 study demonstrated that anti-PEG IgG showed no significant booster effect after each dose, but detected a significant increase in anti-PEG IgM levels after the first and third dose. (Guerrini, G. et al., L. Monitoring Anti-PEG Antibodies Level upon Repeated Lipid Nanoparticle-Based COVID-19 Vaccine Administration. Int. J. Mol. Sci. 23, 8838 (2022)). While the increase in anti-PEG IgM after the first dose is expected since IgMs are involved in early immunological response to infection, the significant increase of anti-PEG-IgM after either dose may compromise patient safety. A 2023 study simulating the clinical practice of Comirnaty® demonstrated the accelerated blood clearance (ABC) phenomenon of clinically relevant LNP (Wang, H. et al., Polyethylene glycol (PEG)-associated immune responses triggered by clinically relevant lipid nanoparticles in rats. npj Vaccines 8, 169 (2023)). In addition, this study demonstrated that induction of anti-PEG IgM and IgG by PEGylated LNP were both time-and dose-dependent and, somewhat contrary to the previous 2022 study, specifically found a higher level of anti-PEG IgM and IgG induced after repeated injection with PEGylated LNP. Regardless of the study results with respect to IgG, it is a consistent finding that anti-PEG IgM increases with repeated injection. Finally, a study investigating whether anti-PEG antibodies are boosted following the approved mRNA vaccines (Pfizer/BioNTech and Moderna) showed that the Moderna vaccine boosted IgG and IgM anti-PEG antibodies 13.1 and 65.8-fold, respectively, compared to the pre-vax antibody levels (Ju, et. al., Anti-PEG Antibodies Boosted in Humans by SARS-COV-2 Lipid Nanoparticle mRNA Vaccine, ACS Nano 16, 11769-11780 (2022)). Of note, the PEG-lipid in the Moderna LNP is PEG-DMG. Anti-PEG IgG and IgM levels following the Pfizer vaccine dosing were boosted only modestly compared to the Moderna vaccine (−1.78 for IgG and 2.64 for IgM). The PEG-lipid in the Pfizer vaccine is PEG-DMA (ALC-0159, Acuitas).

As such, treating patients who already have been exposed to products containing PEG and have pre-formed anti-PEG antibodies and/or administering multiple doses to patients over time (even if such patients do not have pre-formed anti-PEG antibodies) with LNPs containing PEG-lipids may result in accelerated blood clearance of LNPs containing PEG-lipids, reduced/compromised efficacy, hypersensitivity reactions, and, in some cases, severe allergic reactions to PEG. With the large-scale vaccination of PEGylated LNP-based COVID-19 mRNA vaccines, there remains a need for a non-immunogenic delivery system that provides for repeated administration(s) without accelerated blood clearance, reduced/compromised efficacy, hypersensitivity reactions, or severe allergic reactions to PEG.

Indeed, it would be advantageous to identify LNP formulations that have desirable properties for effective delivery of payload, while also avoiding an IgG and IgM response. The PEOZ-lipid LNP of the present disclosure have desirable particle size, polydispersity, freeze/thaw stability, oligonucleotide encapsulation efficiency, maintenance of oligonucleotide integrity, endosomal escape, transfection efficiency and also provides an absent or markedly reduced IgM and IgG response (as compared to a comparable PEG-lipid currently used in LNP vaccine delivery systems).

### SUMMARY

The present disclosure relates to lipid nanoparticles (LNPs) including a PEOZ-lipid conjugate of one of the following Formula I:

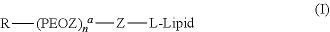

wherein R includes an initiating group,
PEOZ includes a poly(ethyloxazoline) polymer,
L includes a linking group with controllable degradability in physiological media, and n ranges from 1 to 1,000,
a is ran, which indicates a random copolymer, or block, which indicates a block copolymer, and
Z includes S, N, or O, and
Lipid includes N,N-dimethyltetradecylamine (dimyristylamine or DMA).

In some embodiments, the PEOZ-lipid LNPs include a PEOZ-lipid conjugate, a cationic or ionizable lipid, and optional additional lipids. In other embodiments, the PEOZ-lipid LNPs include a PEOZ-lipid conjugate, a cationic POZ, and optional additional lipids. In some aspects, the additional lipids such as phospholipids, structural lipids, cholesterol, and combinations thereof.

The PEOZ-lipid LNP may include a payload. In some aspects, the PEOZ-lipid LNPs include an oligonucleotide payload. In other aspects, the payload is a protein. In still other aspects, the payload is a combination of an oligonucleotide and a protein. The PEOZ-lipid LNPs encapsulate the payload so as to provide for expression of the payload in suitable cell types that take up the LNP, thus providing a therapeutic response to the payload. The oligonucleotide payloads may include, but are not limited to, mRNA vaccines against an infectious disease such as SARS-COV-2, rabies, influenza, and others. The PEOZ-lipid LNPs may also be used in various therapeutic approaches including, but not limited to cancer immunotherapy, gene therapy, enzyme replacement, and combinations thereof.

In some aspects of the present disclosure, the PEOZ-lipid may be

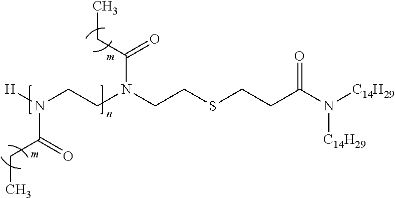

where m is 1, n ranges from 1 to 1000, o ranges from 1 to 5, and p ranges from 1 to 10.

In another embodiment, the present disclosure relates to a method for raising a protective immune response while not raising an IgM or IgG response in an animal, including the step of administering to the animal an effective amount of such PEOZ-lipid LNP compositions. In some aspects, the step of administering may include delivering such PEOZ-lipid LNP compositions to the animal via subcutaneous, intravenous, intramuscular, intradermal or aerosol routes.

The present disclosure also related to a method of delivering a payload in a subject without raising a PEOZ-associated immune response including:
providing a lipid nanoparticle composition including:
   a compound of Formula I

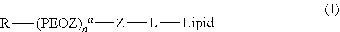

where R includes an initiating group, PEOZ includes a polymer of the structure [N(COR₂)CH₂CH₂], where R₂ is ethyl, n ranges from 1 to 1,000, a is ran, which indicates a random copolymer, or block, which indicates a block copolymer, Z includes S, O, or N, L includes a linking group with controllable degradability in physiological media, and Lipid includes dimyristylamine;
an ionizable or cationic lipid or cationic POZ;
a helper lipid;
a sterol lipid; and
a payload; and
   administering to the subject an effective amount of the lipid nanoparticle composition.

In some aspects, the PEOZ has a molecular weight between 500 Daltons and 5,000 Daltons. In other aspects, the PEOZ has a molecular weight between 1,500 Daltons and 3,000 Daltons. In some embodiments, R includes a hydrogen or a substituted or unsubstituted alkyl. In other embodiments, Formula I is:

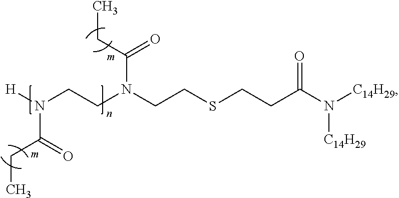

where m is 1, n ranges from 1 to 1000, and p ranges from 1 to 10. In yet other embodiments, L is —CH₂CH_{2—}G—, and wherein G includes the linking group. In still other embodiments, G includes ethers, esters, carboxylate esters, carbonate esters, carbamates, amines, amides, disulfides, or combinations thereof.

R may be a hydrogen, or a substituted or unsubstituted alkyl, and wherein n ranges from 15 to 35. In some respects, the step of administering includes delivering the pharmaceutical composition to the animal via subcutaneous, intravenous, intramuscular, intradermal or aerosol routes. In other respects, the PEOZ-associated immune response includes an IgM response, an IgG response, or a combination thereof.

The present disclosure also relates to a method for raising a protective immune response in an animal without raising a PEOZ-associated immune response, including the steps of:
providing a lipid nanoparticle composition including
   a compound of Formula I

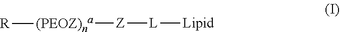

where R includes an initiating group, PEOZ includes a polymer of the structure [N(COR₂)CH₂CH₂], wherein R₂ is ethyl, n ranges from 1 to 1,000, a is ran, which indicates a random copolymer, or block, which indicates a block copolymer, Z includes S, L includes —CH2CH2—G—, and Lipid includes dimyristylamine;
an ionizable lipid;
a helper lipid;
a sterol lipid; and
an oligonucleotide payload, and
   administering to the animal an effective amount of the lipid nanoparticle composition.

In some embodiments, the method further includes repeating the step of administering after a predetermined amount of time. In other embodiments, the PEOZ-associated immune response includes an IgM response, an IgG response, or a combination thereof.

In some aspects, G includes a linking group. In other aspects, the PEOZ has a molecular weight between 500 Daltons and 5,000 Daltons. In yet other aspects, the PEOZ has a molecular weight between 1,500 Daltons and 3,000 Daltons. In still other aspects, R includes a hydrogen or a substituted or unsubstituted alkyl.

In certain embodiments, Formula I is:

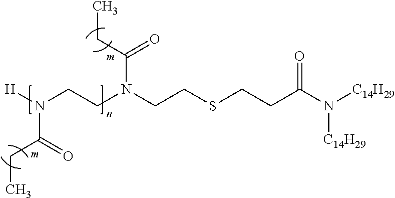

wherein m is 1, n ranges from 1 to 1000, and p ranges from 1 to 10.

In other embodiments, G includes ethers, esters, carboxylate esters, carbonate esters, carbamates, amines, amides, disulfides, or combinations thereof. In yet other embodiments, R includes a hydrogen, or a substituted or unsubstituted alkyl, and wherein n ranges from 15 to 35. In still other embodiments, the step of administering includes delivering the pharmaceutical composition to the animal via subcutaneous, intravenous, intramuscular, intradermal or aerosol routes.

### BRIEF DESCRIPTION OF THE DRAWINGS

Further features and advantages of the invention can be ascertained from the following detailed description that is provided in connection with the drawings described below:

FIG. 1A shows a lipid nanoparticle in accordance with an embodiment of the present disclosure.

FIG. 1B shows a lipid nanoparticle in accordance with another embodiment of the present disclosure.

FIGS. 2A-2B are graphical representations on a normal y-scale and log y-scale, respectively, of the IgM response in rats after administration of LNPs including PEG-DMA and including PEOZ-DMA in accordance with the present disclosure.

FIGS. 3A-3B are graphical representations on a normal y-scale and log y-scale, respectively, of the IgG response in rats after administration of LNPs including PEG-DMA and including PEOZ-DMA in accordance with the present disclosure.

### DETAILED DESCRIPTION

The present disclosure relates to a delivery method for raising a protective immune response while not raising an immune response to the polymer portion of the LNP in an animal. More specifically, the present disclosure relates to a method of delivering a payload to an animal via a PEOZ-lipid LNPs without raising an IgM or IgG response to the PEOZ portion of the LNP.

The PEOZ-lipid LNPs disclosed herein may be used to facilitate the intracellular delivery of biologically active and therapeutic molecules. In some embodiments, the PEOZ-lipid LNPs may be used to deliver an encapsulated payload, e.g., a nucleic acid payload including, but not limited to, mRNA or modified mRNA. Because the PEOZ-lipid LNPs of the present disclosure have no immunogenicity or significantly reduced immunogenicity as compared to a corresponding LNP containing a PEG-lipid, such LNPs provide a safer method of delivering nucleic acid vaccines by reducing or inhibiting the IgM or IgG response to the polymer portion while still allowing for the desired immune response. For example, when the PEOZ-lipid LNPs of the present disclosure are used to deliver antigenic mRNA encoding a nucleoside-modified prefusion form of the spike antigen (S-2P), the desired immune response includes robust production of spike-binding and neutralizing antibodies, as well as intermediate levels of T-cell responses, but there is little to no immune response to the PEOZ portion of the LNP.

The disclosure also relates to pharmaceutical compositions that include such PEOZ-lipid LNPs and that are useful to deliver therapeutically effective amounts of biologically active molecules into the cells of patients.

### Definitions

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The articles “a” and “an” are used herein to refer to one or to more than one (i.e., to at least one) of the grammatical object of the article. By way of example, “an element” means one element or more than one element.

The terms “about” and “approximately” shall generally mean an acceptable degree of error or variation for the quantity measured given the nature or precision of the measurements. Numerical quantities given in this description are approximate unless stated otherwise, meaning that the term “about” or “approximately” can be inferred when not expressly stated.

As used herein, the term “active” or “activated” when used in conjunction with a particular functional group refers to a functional group that reacts readily with an electrophile or a nucleophile on another molecule. This is in contrast to those groups that require catalysts or impractical reaction conditions in order to react (i.e., a “non-reactive” or “inert” group).

As used herein, the term “physiologically degradable” or “physiologically releasable” refers to a linkage containing a cleavable moiety. The terms degradable and releasable do not imply any particular mechanism by which the linker is cleaved.

As used herein, the term “link”, “linked” “linkage” or “linker” when used with respect to a PEOZ polymer, PEOZ conjugate, an agent, or compound described herein, or components thereof, refers to bonds that normally are formed as the result of a chemical reaction and typically are covalent linkages.

As used herein, the term “lipid nanoparticle” or “LNP” is used to encompass any of the many types of nanoparticles, including liposomes, that are formed by a lipid layer or layers surrounding a core containing a molecule to be released into the body. Liposomes generally have one or more contiguous lipid bilayers encapsulating an aqueous core. Other forms of liposome-like nanocarriers may have a lipid monolayer, or a non-contiguous bilayer, and may or may not have an aqueous core.

As used herein, the term “hydrophilic”, for example with reference to a hydrophilic group, refers to a compound or molecule, or a portion thereof, where the interaction with water is thermodynamically more favorable than interaction with oil or other hydrophobic solvents. A hydrophilic compound is able to dissolve in, or be dispersed in, water.

As used herein, the term “hydrophobic”, for example with reference to a hydrophobic portion, refers to a compound or molecule, or a portion thereof, where the interaction with water is thermodynamically less favorable than interaction with oil or other hydrophobic solvents. A hydrophobic compound is able to dissolve in, or be dispersed in, oil or other hydrophobic solvents.

As used herein, the term “inert” or “non-reactive” when used in conjunction with a particular functional group refers to a functional group that does not react readily with an electrophile or a nucleophile on another molecule and require catalysts or impractical reaction conditions in order to react.

As used herein, the term “pendent group” refers to a part of the PEOZ polymer that is attached to the PEOZ polymer.

As used herein, the term “pendent moiety” refers to a substituent that is linked to the POZ polymer portion via a linking group; a pendent moiety is exemplified by R₂ of formula IV as described herein.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable form” is meant to include known forms of a compound or POZ conjugate that may be administered to a subject, including, but not limited to, solvates, hydrates, prodrugs, isomorphs, polymorphs, pseudomorphs, neutral forms and salt forms of a compound. In certain embodiments, the pharmaceutically acceptable form excludes prodrugs, isomorphs and/or pseudomorphs. In certain embodiments, the pharmaceutically acceptable form is limited to pharmaceutically acceptable salts, neutral forms, solvates and hydrates. In certain embodiments, the pharmaceutically acceptable form is limited to pharmaceutically acceptable salts and neutral forms. In certain embodiments, the pharmaceutically acceptable form is limited to pharmaceutically acceptable salts.

As used herein, the term “alkyl”, whether used alone or as part of a substituent group, is a term of art and refers to saturated aliphatic groups that optionally contain one or more heteroatoms (such as O, S or N) which may be optionally substituted, including straight-chain alkyl groups, branched-chain alkyl groups, cycloalkyl groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C10 straight-chain alkyl group or a C₁-C₃ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl. In certain embodiments, the term “alkyl” refers to a C₁-C₁₀ straight-chain alkyl group that contains one or more heteroatoms in place of a carbon atom (such as O, S or N), wherein the heteroatom may be optionally substituted. In certain embodiments, the term “alkyl” refers to a C₁-C₁₀ straight-chain alkyl group that is substituted with up to 5 groups selected from the group consisting of OH, NH₂ and ═O.

As used herein, the term “alkenyl”, whether used alone or as part of a substituent group, is a term of art and refers to unsaturated aliphatic groups that optionally contain one or more heteroatoms (such as O, S or N) which may be optionally substituted, including, a straight or branched chain hydrocarbon radical containing from 2 to 30 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

As used herein, the term “alkynyl”, whether used alone or as part of a substituent group, is a term of art and refers to unsaturated aliphatic groups that optionally contain one or more heteroatoms (such as O, S or N) which may be optionally substituted, including, straight or branched chain hydrocarbon radical containing from 2 to 30 carbon atoms and containing at least one carbon-carbon triple bond. Representative examples of alkynyl include, but are not limited, to acetylenyl, 1-propynyl, 2-propynyl, 3-butynyl, 2-pentynyl, 4-pentynyl, and 1-butynyl.

As used herein, the term “substituted alkyl”, “substituted alkenyl”, and “substituted alkynyl” refers to alkyl, alkenyl and alkynyl groups as defined above in which one or more bonds to a carbon(s) or hydrogen(s) are replaced by a bond to non-hydrogen or non-carbon atoms such as, but not limited to, a halogen atom in halides such as F, Cl, Br, and I; and oxygen atom in groups such as carbonyl, carboxyl, hydroxyl groups, alkoxy groups, aryloxy groups, heterocyclyloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines imines, oximes, hydrazones, heterocyclylamine, (alkyl)(heterocyclyl)-amine, (aryl)(heterocyclyl) amine, diheterocyclylamine, triazoles, and nitriles; a silicon atom in groups such as in trialkyl silyl groups, dialkylarylsilyl groups, alkyldiarylsilyl groups, and triarylsilyl groups; and other heteroatoms in various other groups. In a specific embodiment, a “polar alkyl”, “polar alkenyl”, and “polar alkynyl”, refers to alkyl, alkenyl, and alkynyl groups substituted with an atom that results in a polar covalent bond. In another specific embodiment, a “polar alkyl”, “polar alkenyl”, and “polar alkynyl” refers to C1 to C5 alkyl, alkenyl, and alkynyl, groups substituted with an atom that results in a polar covalent bond. In a specific embodiment, a “polar alkyl”, “polar alkenyl”, and “polar alkynyl”, refers to alkyl, alkenyl, alkynyl groups, such as C1 to C5 alkyl, alkenyl, and alkynyl groups, substituted with an —OH group and/or a —C(O)—OH group.

As used herein, the term “halo” or “halogen” whether used alone or as part of a substituent group, is a term of art and refers to —F, —Cl, —Br, or —I.

As used herein, the term “alkoxy”, whether used alone or as part of a substituent group, is a term of art and refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “aralkyl” or “arylalkyl”, whether used alone or as part of a substituent group, is a term of art and refers to an alkyl group substituted with an aryl group, wherein the moiety is appended to the parent molecule through the alkyl group. An arylalkyl group may be optionally substituted. A “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom.

As used herein, the term “heteroaralkyl” or “heteroarylalkyl”, whether used alone or as part of a substituent group, is a term of art and refers to an alkyl group substituted with a heteroaryl group, wherein the moiety is appended to the parent molecular moiety through the alkyl group. A heteroarylalkyl may be optionally substituted. The term “substituted heteroarylalkyl” has the same meaning with respect to unsubstituted heteroarylalkyl groups that substituted aryl groups had with respect to unsubstituted aryl groups.

As used herein, the term “heterocyclylalkyl”, whether used alone or as part of a substituent group, is a term of art and refers to unsubstituted or substituted alkyl, alkenyl or alkynyl groups in which a hydrogen or carbon bond of the unsubstituted or substituted alkyl, alkenyl or alkynyl group is replaced with a bond to a heterocyclyl group. A heterocyclylalkyl may be optionally substituted. The term “substituted heterocyclylalkyl” has the same meaning with respect to unsubstituted heterocyclylalkyl groups that substituted aryl groups had with respect to unsubstituted aryl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

As used herein, the term “aryl”, whether used alone or as part of a substituent group, is a term of art and refers to includes monocyclic, bicyclic and polycyclic aromatic hydrocarbon groups, for example, benzene, naphthalene, anthracene, and pyrene. The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In certain embodiments, the term “aryl” refers to a phenyl group. The aryl group may be optionally substituted.

As used herein, the term “cycloalkyl”, whether used alone or as part of a substituent group, is a term of art and refers to a saturated carbocyclic group containing from three to six ring carbon atoms, wherein such ring may optionally be substituted with a substituted or unsubstituted alkyl group or a substituent as described for a substituted alkyl group. Exemplary cycloalkyl groups include, but are not limited to, cyclopropyl, cyclobutyl, cyclopentyl, cyclohexyl, 2-methylcyclobutyl and 4-ethylcyclohexyl.

As used herein, the term “heteroaryl”, whether used alone or as part of a substituent group, is a term of art and refers to a monocyclic, bicyclic, and polycyclic aromatic group having 3 to 30 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

As used herein, the term “heterocyclyl”, whether used alone or as part of a substituent group, is a term of art and refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 15 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocyclyl group may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like.

As used herein, the terms “treatment”, “treat”, and “treating” refers a course of action (such as administering a conjugate as described herein or pharmaceutical composition comprising a conjugate as described herein) so as to prevent, eliminate, or reduce a symptom, aspect, or characteristics of a disease or condition. Such treating need not be absolute to be useful. In one embodiment, treatment includes a course of action that is initiated concurrently with or after the onset of a symptom, aspect, or characteristics of a disease or condition. In another embodiment, treatment includes a course of action that is initiated before the onset of a symptom, aspect, or characteristics of a disease or condition.

As used herein, the term “in need of treatment” refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease or condition that is treatable by a method or compound of the disclosure.

As used herein, the terms “individual”, “subject”, or “patient” refers to any animal, including mammals, such as mice, rats, other rodents, rabbits, dogs, cats, swine, cattle, sheep, horses, or primates, and humans. The terms may specify male or female or both, or exclude male or female. In a preferred embodiment, the terms “individual”, “subject”, or “patient” refers to a human.

As used herein, the term “therapeutically effective amount” refers to an amount of a conjugate, either alone or as a part of a pharmaceutical composition, that is capable of having any detectable, positive effect on any symptom, aspect, or characteristics of a disease or condition. Such effect need not be absolute to be beneficial.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

It will be understood that when a group is specified as a part of a compound, the substitution of the group may be adjusted to accommodate the particular bonds. For example, when an alkyl group is joined to two other groups, the alkyl group is considered an alkylene group.

The term “substituted” is also contemplated to include all permissible substituents of organic compounds. In a broad aspect, the permissible substituents include acyclic and cyclic, branched and unbranched substituents, carbocyclic and heterocyclyl, aromatic and nonaromatic substituents of organic compounds. Illustrative substituents include, for example, those described herein above. For purposes of this disclosure, the heteroatoms, such as oxygen or nitrogen, may have hydrogen substituents and/or any permissible substituents of organic compounds described herein which satisfy the valences of the heteroatoms. Exemplary substitutions include, but are not limited to, hydroxy, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. This invention is not intended to be limited in any manner by the permissible substituents of organic compounds.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of conjugate. As another example, the salt may comprise less than one inorganic or organic acid molecule per molecule of base, such as two molecules of conjugate per inorganic or organic acid molecule.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, iso osmotic, cryo-preservatives, lubricating, flavoring, and coloring agents may be used. Other examples of suitable pharmaceutical carriers are described in Remington's Science and Practice of Pharmacy (23^{rd} edition, ISBN 9780128200070) and Handbook of Pharmaceutical Excipients (8^{th} edition, 978-0-85-711271-2), each herein incorporated by reference in their entirety.

As used herein, the term “target molecule” refers to any molecule having a therapeutic or diagnostic application or a targeting function, or a vehicle with which a compound is administered or formulated for administration, wherein the target molecule is capable of forming a linkage with an active functional group on a POZ polymer or a POZ derivative of the present disclosure, including, but not limited to, a therapeutic agent (such as but not limited to a drug), a diagnostic agent, a targeting agent, an organic small molecule, an oligonucleotide, a polypeptide, an antibody, an antibody fragment, a protein, a carbohydrate such as heparin or hyaluronic acid, or a lipid such as a glycerolipid, glycolipid, or phospholipid.

As used herein, “lipid” or “lipid portion” means (i) an organic compound that includes an ester of fatty acid or a derivative thereof and is characterized by being insoluble in water, but soluble in many organic solvents, and includes, but is not limited to, simple lipids such as fats, oils, and waxes, compound lipids such as phospholipids, glycolipids, cationic lipids, non-cationic lipids, neutral lipids, and anionic lipids, and derived lipids such as steroids, as well as (ii) an organic compound that does not include an ester of fatty acid, but mimics such an organic compound through its amphipathic character, i.e., it possesses both hydrophobic and hydrophilic portions, and, thus, is able to aggregate in a specific manner to form layers, vesicles and LNPs in aqueous environments.

As used herein, “small interfering RNA (siRNA)” mean a class of double-stranded RNA molecules, 16-40 nucleotides in length, that are involved in the RNA interference (RNAi) pathway, where it interferes with the expression of a specific gene. In addition to their role in the RNAi pathway, siRNAs also act in RNAi-related pathways.

As used herein, “RNA” means a molecule comprising at least one ribonucleotide residue, including siRNA, antisense RNA, single stranded RNA, microRNA, mRNA, noncoding RNA, and multivalent RNA. “Ribonucleotide” means a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides.

### PEOZ-Lipid LNPs

The PEOZ-lipid LNPs of the present disclosure are substantially non-immunogenic. More specifically, unlike its PEG-lipid counterpart, when incorporated into LNP compositions as described herein, the PEOZ-lipid conjugate do not generate a significant immune response to PEOZ. In one aspect, the PEOZ-lipid LNPs do not generate IgM and/or IgG antibodies specific to PEOZ. In another aspect, PEOZ-lipid LNPs of the present disclosure generate a reduced immune response, including, but not limited to, the generation of IgM and/or IgG antibodies specific to the PEOZ (as compared to a corresponding LNP composition incorporating a PEG-lipid conjugate).

In still another aspect, after a second or repeated administration of a PEOZ-lipid LNP of the present disclosure, the PEOZ-lipid LNP is present in the blood or a tissue of the subject at a concentration of at least 75%, such as 80%, 85%, 90%, 95%, or greater, as compared to the first administration. In another aspect, a PEOZ-lipid LNP of the present disclosure is not subject to accelerated blood clearance (ABC).

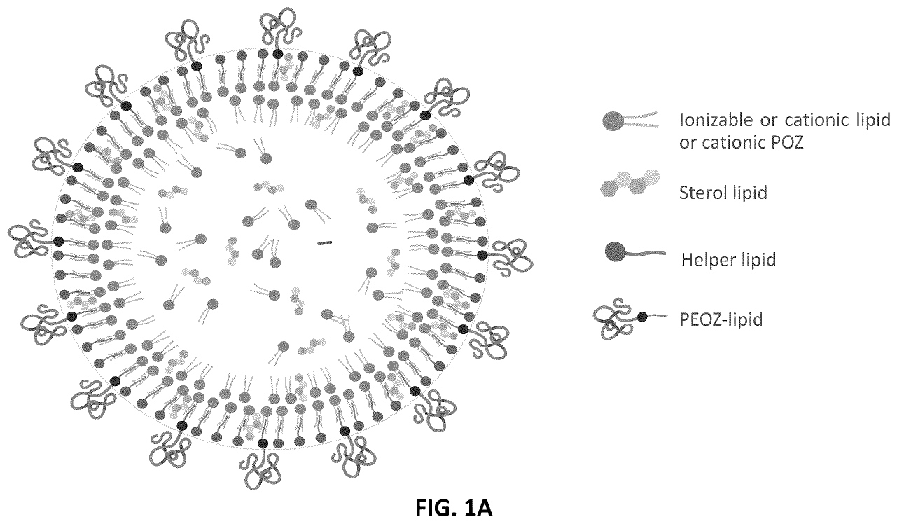

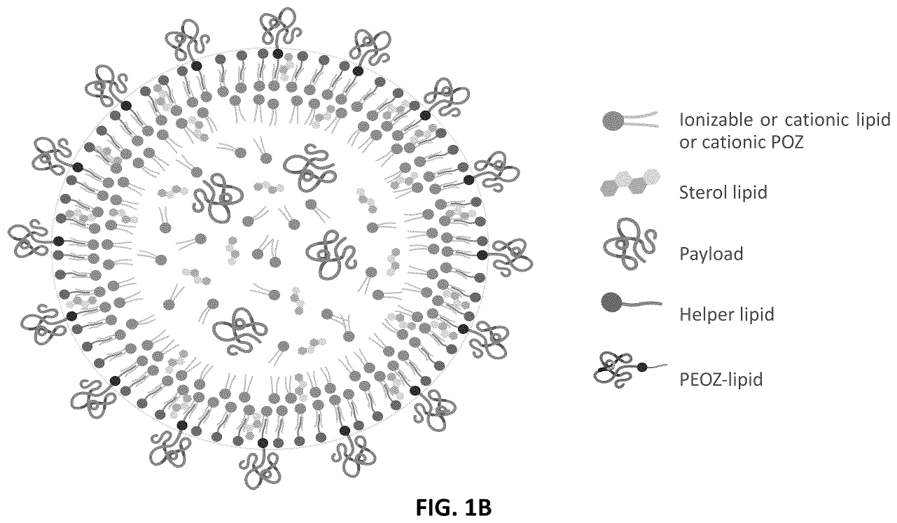

The PEOZ-lipid LNPs are amphiphilic spherical vesicles formed by one or more lipid layers enveloping an aqueous core with size ranging from about 20 nm to a few microns. As shown in FIG. 1A, PEOZ-lipid LNPs of the present disclosure include (i) a PEOZ-lipid to provide the LNP with a hydrating layer to improve colloidal stability, prevent fusion of nascent particles, reduce protein adsorption and non-specific uptake, and prevent reticuloendothelial clearance; (ii) a cationic or ionizable lipid or cationic POZ; (iii) a helper lipid that supports the bilayer structure and facilitates the endocytosis; and (iv) a sterol lipid (i.e., cholesterol) to stabilize the lipid bilayer of the LNP. The PEOZ-lipid LNPs of the present disclosure may be prepared by a variety of methods. Suitable methods includes those described in U.S. Patent Publication No. 2022/0249695, the entire disclosure of which is incorporated by reference herein. The components and properties of the PEOZ-lipid LNPs of the present disclosure are discussed in more detail below.

### PEOZ-Lipid

In some embodiments, the PEOZ-lipid may be a PEOZ-lipid of Formula I:

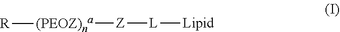

where R₁ includes an initiating group, PEOZ comprises poly (ethyloxazoline), n ranges from 1 to 1,000; a is ran, which indicates a random copolymer, or block, which indicates a block copolymer; and Z comprises S, O, or N; L includes a physiologically degradable linking group, and Lipid includes N,N-dimethyltetradecylamine (dimyristylamine or DMA). In some aspects, PEOZ includes [N(COR₂)CH₂CH₂]ₙ, where R₂ is ethyl. In still other embodiments, R₁ includes a hydrogen, a substituted or unsubstituted alkyl (such as a C1 to C4 alkyl group), an alkyne-substituted alkyl, a triazole with attached carboxylic acid, or a substituted or unsubstituted aralkyl group. In some embodiments, R is H or CH₃. In another embodiment, the initiating group is H. In some aspects, the initiating group may be selected to lack an active functional group. In other aspects, the initiating group may be selected to include an active functional group. Additional suitable initiating groups are disclosed in U.S. Pat. Nos. 7,943,141, 8,088,884, 8,110,651, 8,101,706, 8,883,211, and 9,284,411, and U.S. patents application Ser. Nos. 13/003,306, 13/549,312 and 13/524,994, each of which is incorporated by reference in its entirety for such teachings.

In some embodiments, L is a linking group containing a cleavable moiety in which the rate of cleavage is controlled and represents a direct linkage through a reactive group on the lipid and a reactive group on the polymer, wherein the direct linkage may form a cleavable moiety in which the rate of cleavage can be controlled from highly labile to stable. In yet other embodiments, L includes ethers, esters, carboxylate esters (—C(O)—O), carbonate esters (—O—C(O)—O—), carbamates (—O—C(O)—NH—), amines, amides (—C(O)—NH—), urethanes, disulfides, and combinations thereof.

In one aspect, the PEOZ in Formula I may be a polymer represented by [N(COR₂)CH₂CH₂]ₙ, wherein R₂ is ethyl, R is H or CH3, and the degree of polymerization “n” may range from 15 to 35, 20 to 30, or 25.

Specific embodiments of the foregoing Formula I include, but are not limited to, L being an amidase-cleavable amide as shown below in I(a), I(b), and I(c):

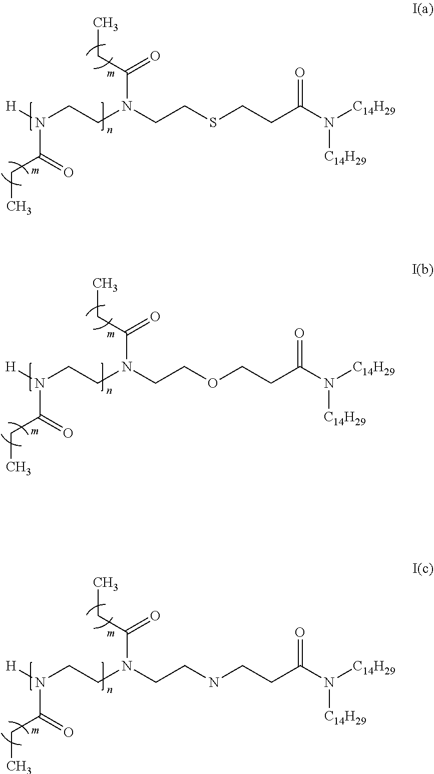

In one embodiment, m is 1, n ranges from I to 1000 for the Formulas I(e)(1) and I(e)(2).

More specific embodiments and examples of suitable PEOZ lipids for use in a PEOZ-lipid LNP made in accordance with the present disclosure are provided in U.S. Patent Publication No. 2022/0249695.

In some aspects, L contains a linkage that is physiologically degradable in that it can be cleaved in specific environments. For example, the linkage may be cleaved in vivo in a subject after administration of a PEOZ-lipid LNP of the present disclosure to the subject. In one embodiment, the cleavable moiety is cleaved by a chemical reaction such as by reduction of an easily reduced group (e.g., a disulfide). In another embodiment, the cleavable moiety is cleaved by a substance that is naturally present or induced to be present in the subject such as an enzyme or polypeptide. In yet another embodiment, the cleavable moiety is cleaved by a combination of the foregoing. The linking group may contain portions of the PEOZ polymer and/or portions of the lipid as such portions have reacted to form the linking group as discussed below.

In this aspect, suitable cleavable moieties include, but are not limited to, esters, carboxylate esters (—C(O)—O—), carbonate esters (—O—C(O)—O—), carbamates (—O—C(O)—NH—) and amides (—C(O)—NH—, including an amide group in a peptide); other releasable moieties are discussed herein. In a particular embodiment, the cleavable moiety is an ester. In another particular embodiment, the cleavable moiety is a carbonate ester or a carboxylate ester. In addition, the linking group may be a naturally occurring amino acid, a non-naturally occurring amino acid or a polymer containing one or more naturally occurring and/or non-naturally occurring amino acids. The linking group may include certain groups from the polymer chain and/or the lipid.

The ratio of PEOZ-lipid to the other components in the PEOZ-lipid LNP may be varied in order to allow for optimal size, high payload release and transfection, and improved stability of the hydrated formulation. In one embodiment, the mol percent of PEOZ-lipid in the PEOZ-lipid LNP is about 0.5 to 60 percent. In another embodiment, the mol percent of PEOZ-lipid is about 1 to about 40 percent. In still another embodiment, the PEOZ-lipid is present in an amount of about less than 10 mol percent of the total amount of lipids in the PEOZ-lipid LNP. In this aspect, the PEOZ-lipid may be present in an amount of about 0.5 to about 5 mol percent, about 1 to about 4 mol percent, or about 1.5 to about 3.5 mol percent. In this aspect, the remainder of the PEOZ-lipid LNP composition may be about 35 to about 50 mol percent sterol lipid, about 30 percent to about 70 mol percent cationic or ionizable lipid or cationic POZ, and about 5 percent to about 15 mol percent helper lipid.

In some embodiments, the PEOZ-lipid conjugate is present at a mole ratio of about 0.25% to about 5% mole percent in the lipid layer of the PEOZ-lipid LNP, at a mole ratio of about 0.5% to about 3% mole percent in the lipid layer of the PEOZ-lipid LNP, at a mole ratio of about 0.75% to about 2% mole percent in the lipid layer of the PEOZ-lipid LNP, or at a mole ratio of about 0.8% to about 1.5% mole percent in the lipid layer of the PEOZ-lipid LNP.

### Ionizable and Cationic Lipids

Suitable ionizable lipids include, but are not limited to, MC3 98, Lipid 319, C12-200, 5A2-SC8, 306Oi10, Moderna Lipid 5, Moderna Lipid H, SM-102, Acuitas A9 [59], Arcturus Lipid 2,2 (8,8) 4C CH3, Genevant CL1. A non-limiting example of a cationic lipid suitable for use in accordance with the present invention is 1,2-dioleoyl-3-trimethylammonium propane (DOTAP). In one embodiment, the cationic or ionizable lipids have a pKa, as measured by the TNS dye-binding assay, in the range of 6-7.

### Cationic POZ

In some embodiments, the PEOZ-lipid LNPs of the present disclosure include a cationic POZ in place of the conventionally employed ionizable or cationic lipid. Suitable cationic POZ are described in U.S. patent application Ser. No. 18/743,721, filed Jun. 14, 2024, the entire disclosure of which is incorporated by reference herein. In some embodiments, the cationic POZ is cationic poly (oxazoline) (cationic POZ). In some aspects, the cationic POZ may be a POZ-cation of Formula II(a):

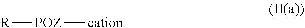

where R includes an initiating group and POZ is a polyoxazoline polymer. In some aspects, R includes a hydrogen, a substituted or unsubstituted alkyl, an alkyne-substituted alkyl, or a substituted or unsubstituted aralkyl group. In other aspects, POZ may be poly(ethyloxazoline). In still other aspects, POZ may be a polyoxazoline polymer of the structure [N(COR₂)CH₂CH₂]ₙ where R₂ is ethyl and n ranges from 1 to 1000.

In other embodiments, the cationic POZ may be a POZ-cation of Formula II(b):

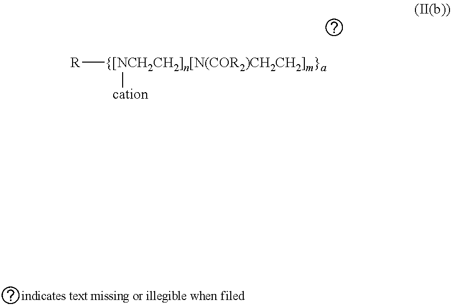

where R includes an initiating group, n ranges from 1 to 10, R₂ is independently selected for each repeating unit from an unsubstituted or substituted alkyl, alkenyl, aralkyl, heterocyclylalkyl, or active functional group, m ranges from 1 to 100, a is ran, which indicates a random copolymer, or block, which indicates a block copolymer, and T includes a terminating group. In some aspects, R includes a hydrogen, a substituted or unsubstituted alkyl, an alkyne-substituted alkyl, a triazole with attached carboxylic acid, or a substituted or unsubstituted aralkyl group.

In other embodiments, the cationic POZ may be a POZ-cation of Formula II(c):

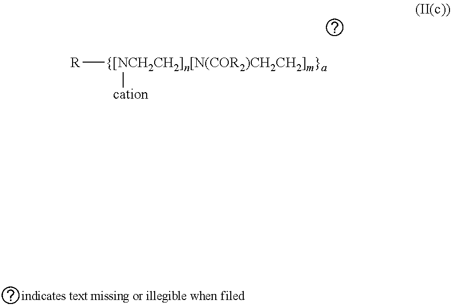

where R includes an initiating group, n ranges from 1 to 10, R₂ is independently selected for each repeating unit from an unsubstituted or substituted alkyl, alkenyl, aralkyl, heterocyclylalkyl, or active functional group, m ranges from 1 to 100, a is ran, which indicates a random copolymer, or block, which indicates a block copolymer, and T includes a terminating group. In some aspects, R includes a hydrogen, a substituted or unsubstituted alkyl, an alkyne-substituted alkyl, a triazole with attached carboxylic acid, or a substituted or unsubstituted aralkyl group.

In still other embodiments, the cationic POZ may be a POZ-cation of Formula II(d):

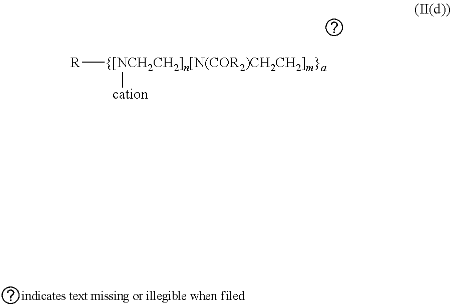

where R includes a branching group, n ranges from 1 to 10, R₂ is independently selected for each repeating unit from an unsubstituted or substituted alkyl, alkenyl, aralkyl, heterocyclylalkyl, or active functional group, m ranges from 1 to 100, a ranges from 3 to 20, and T includes a terminating group. In some aspects, R includes glycerol, pentaerythritol, polyglycerol, etc.

### Helper Lipid

Non-limiting examples of helper lipids suitable for use in accordance with the present disclosure include phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length and have varying degrees of unsaturation. Other suitable helper lipids include, but are limited to, glycolipids, such as cerebrosides and gangliosides. In one aspect, the helper lipid is at least one of 1,2-dioleoyl-sn-glycero-3-phosphoethanolamine (DOPE), 1,2-distearoyl-sn-glycero-3-phosphocholine (DSPC), and POPE (1-palmitoyl-2-oleoylsn-glycero-3-phosphoethanolamine).

### Sterol Lipid

A suitable sterol lipid for use in accordance with the present disclosure is cholesterol. Another suitable sterol lipid for use in accordance with the present disclosure is phytosterol.

### Payload

PEOZ-lipid LNPs formed in accordance with the present disclosure may also include a payload. In this aspect, the payload may be an oligonucleotide, protein, or a combination thereof. For example, in a specific embodiment shown in FIG. 1B, PEOZ-lipid LNPs of the present disclosure may include (i) an ionizable or cationic lipid or cationic POZ; (ii) a helper lipid; (iii) a sterol lipid; (iii) a PEOZ-lipid; and (iv) a payload. In another specific embodiment (not shown), LNPs of the present disclosure may include a cationic lipid, a helper lipid, a sterol lipid, a PEOZ-lipid, and an oligonucleotide. In one embodiment, the oligonucleotide comprises DNA, siRNA, self-replicating mRNA, mRNA comprised of modified nucleosides, and mRNA comprised of naturally occurring nucleosides. In one aspect, the oligonucleotide is DNA. In another aspect, the oligonucleotide is siRNA. In still another aspect, the oligonucleotide is self-replicating mRNA, mRNA comprised of modified nucleosides, or mRNA comprised of naturally occurring nucleosides. In yet another aspect, the oligonucleotide is a sgRNA used in genome editing.

In yet another specific embodiment (not shown), PEOZ-lipid LNPs of the present disclosure may include a cationic or ionizable lipid or cationic POZ, a helper lipid, a sterol lipid, a PEOZ-lipid, and a protein.

The payload can be encapsulated into the PEOZ-lipid LNP with high efficiency. In one embodiment, the payload is encapsulated into the PEOZ-lipid LNP with an efficiency of at least 90 percent. In another embodiment, the payload is encapsulated into the PEOZ-lipid LNP with an efficiency of about 90 to about 99 percent. In still another embodiment, the payload is encapsulated into the PEOZ-lipid LNP with an efficiency of about 90 to about 95 percent. In yet another embodiment, the payload is encapsulated into the PEOZ-lipid LNP with an efficiency of greater than about 95 percent.

### Properties

It is important to note that changes or additions (even very minor) to the PEOZ-lipid LNPs of the present disclosure may impact not only the structure of the PEOZ-lipid LNP, but also the delivery of the encapsulated payload. For example, when a sterol lipid such as cholesterol is included in a PEOZ-lipid LNP along with an ionizable lipid and a PEOZ-lipid, the resulting PEOZ-lipid LNP has a single bilayer. If phytosterol is added, the structure of the PEOZ-lipid LNP becomes more complex and, thus, may deliver the payload differently. In this vein, PEOZ-lipid LNPs of the present disclosure including the POZ-lipid conjugates may be unilamellar or non-unilamellar.

The particle size of PEOZ-lipid LNPs made in accordance with the present disclosure can vary. In one embodiment, PEOZ-lipid LNPs formed in accordance with the present disclosure are amphiphilic spherical vesicles formed by one or more lipid bilayers enveloping an aqueous core with size ranging from about 10 nm to about 10 microns. In another embodiment, a PEOZ-lipid LNP formed in accordance with the present disclosure has a particle size of about 25 nm to about 8 microns. In yet another embodiment, a PEOZ-lipid LNP formed in accordance with the present disclosure has a particle size of about 30 nm to about 5 microns. In this aspect, the particle size of the PEOZ-lipid LNP may be between about 20 nm to about 3 microns. In another embodiment, the PEOZ-lipid LNP may be between about 10 nm and about 1000 nm, between about 25 nm and about 500 nm, between about 35 nm and about 250 nm, between about 40 nm and about 150 nm, or between about 45 nm and about 100 nm. Methods of size fractionation are disclosed herein. However, in certain aspects, size fractionation is not required.

### Administration

The PEO-lipid LNPs of the present disclosure may be delivered to a cell. After in vivo administration of the PEOZ-lipid LNPs, the payload is released. In this aspect, the PEOZ-lipid LNPs of the present disclosure may be included a pharmaceutical composition capable of eliciting a treatment for a disorder or disease. For example, pharmaceutical compositions including PEOZ- lipid LNPs made in accordance with the present disclosure may be used to prevent or treat infectious diseases including, but not limited to, SARS-COV-2, rabies, influenza, and others. In addition, pharmaceutical compositions including PEOZ-lipid LNPs made in accordance with the present disclosure may be used as therapeutics for cancer and genetic diseases. Such pharmaceutical compositions may also include a pharmaceutically acceptable carrier in addition to the PEOZ-lipid LNPs.

In one embodiment, a pharmaceutical composition including an effective amount of PEOZ-lipid LNP of the present disclosure can be delivered to an animal. In this aspect, delivery of an effective amount of a LNP of the present disclosure may occur via subcutaneous, intravenous, intramuscular, intradermal or aerosol routes. In one embodiment, the animal is a human.

Importantly, administration of the PEOZ-lipid LNPs of the present disclosure do not generate a significant immune response, including, but not limited to, the generation of IgM or IgG antibodies specific to PEOZ, as compared to the immune response generated by comparable PEG-LNPs. More specifically, the PEOZ-lipid LNPs described herein generate a significantly reduced immune response, including, but not limited to, the generation of IgM or IgG antibodies specific to the polymer portion, as compared to a comparable PEG-lipid LNP.

In some embodiments, a pharmaceutical composition including an effective amount of PEOZ-lipid LNP of the present disclosure may be administered intravenously to deliver an encapsulated payload to any variety of cells including, but not limited to, endothelial cells, Kupffer cells, hepatocyte cells in the liver, macrophages, B cells, T cells, and/or dendritic cells in the spleen without raising an anti-PEOZ immune response. In this aspect, a pharmaceutical composition including an effective amount of PEOZ-lipid LNP of the present disclosure may be administered intravenously to deliver an encapsulated payload without generating IgM antibodies specific to the polymer portion. Without being bound by any particular theory, it is believed that, not only does a PEOZ-lipid LNP of the present disclosure effectively deliver an encapsulated payload after intravenous injection, it does so with significantly less of an IgM or IgG response to the polymer than its PEG-lipid LNP counterpart. In some respects, the IgM or IgG response to the polymer after administration of a pharmaceutical composition including an effective amount of PEOZ-lipid LNP of the present disclosure is less than 10 percent higher than the IgM or IgG prior to administration. In some embodiments, the IgM or IgG response to the polymer after administration of a pharmaceutical composition including an effective amount of PEOZ-lipid LNP of the present disclosure is less than 5 percent higher than the IgM or IgG prior to administration. In other embodiments, the IgM or IgG response to the polymer after administration of a pharmaceutical composition including an effective amount of PEOZ-lipid LNP of the present disclosure is less than 2 percent higher than the IgM or IgG prior to administration. In still other embodiments, the IgM or IgG response to the polymer after administration of a pharmaceutical composition including an effective amount of PEOZ-lipid LNP of the present disclosure is less than 1 percent higher than the IgM or IgG prior to administration. In yet other embodiments, the IgM or IgG response to the polymer after administration of a pharmaceutical composition including an effective amount of PEOZ-lipid LNP of the present disclosure is less than 0.1 percent higher than the IgM or IgG prior to administration.

In other embodiments, a pharmaceutical composition including an effective amount of PEOZ-lipid LNP of the present disclosure may be administered intramuscularly to deliver an encapsulated payload to on-target tissue such as muscle and lumbar aortic lymph nodes, as well as the liver and spleen, without raising an anti-PEOZ immune response. In some embodiments, intramuscular administration of the PEOZ-lipid LNP of the present disclosure facilitates delivery of an encapsulated payload to macrophages, dendritic cells, endothelial cells, and/or fibroblasts in muscle. In other embodiments, intramuscular administration of the PEOZ-lipid LNP of the present disclosure facilitates delivery of an encapsulated payload to macrophages, monocytes, B cells, and/or dendritic cells in muscle without raising an anti-PEOZ immune response. In still other embodiments, intramuscular administration of the PEOZ-lipid LNP of the present disclosure facilitates delivery of an encapsulated payload to endothelial cells, dendritic cells, Kupffer cells, and/or hepatocyte cells in the liver without raising an anti-PEOZ immune response. In yet other embodiments, intramuscular administration of the LNP of the present disclosure facilitates delivery of the encapsulated payload to macrophages, B cells, T cells, and/or dendritic cells in the spleen without raising an anti-PEOZ immune response. Without being bound by any particular theory, it is believed that, not only does a PEOZ-lipid LNP of the present disclosure effectively deliver an encapsulated payload after intramuscular administration, it does so with significantly less of an IgM or IgG response to the polymer than its PEG-lipid LNP counterpart. In some respects, the IgM or IgG response to the polymer after intramuscular administration of a pharmaceutical composition including an effective amount of PEOZ-lipid LNP of the present disclosure is less than 10 percent higher than the IgM or IgG prior to administration. In some embodiments, the IgM or IgG response to the polymer after intramuscular administration of a pharmaceutical composition including an effective amount of PEOZ-lipid LNP of the present disclosure is less than 5 percent higher than the IgM or IgG prior to administration. In other embodiments, the IgM or IgG response to the polymer after intramuscular administration of a pharmaceutical composition including an effective amount of PEOZ-lipid LNP of the present disclosure is less than 2 percent higher than the IgM or IgG prior to administration. In still other embodiments, the IgM or IgG response to the polymer after intramuscular administration of a pharmaceutical composition including an effective amount of PEOZ-lipid LNP of the present disclosure is less than 1 percent higher than the IgM or IgG prior to administration. In yet other embodiments, the IgM or IgG response to the polymer after intramuscular administration of a pharmaceutical composition including an effective amount of PEOZ-lipid LNP of the present disclosure is less than 0.1 percent higher than the IgM or IgG prior to administration.

In some aspects, the PEOZ-lipid LNPs of the present disclosure allow for repeated delivery of an encapsulated payload to target tissues of a subject without producing an immune response to the PEOZ polymer that promotes accelerated blood clearance (ABC) in response to subsequent doses of the LNP. For example, the IgM or IgG response to the polymer after a second or repeated intravenous or intramuscular administration of a pharmaceutical composition including an effective amount of PEOZ-lipid LNP of the present disclosure is less than 10 percent higher than the IgM or IgG prior to administration. In some embodiments, the IgM or IgG response to the polymer after a second or repeated intravenous or intramuscular administration of a pharmaceutical composition including an effective amount of PEOZ-lipid LNP of the present disclosure is less than 5 percent higher than the IgM or IgG prior to administration. In other embodiments, the IgM or IgG response to the polymer after a second or repeated intravenous or intramuscular administration of a pharmaceutical composition including an effective amount of PEOZ-lipid LNP of the present disclosure is less than 2 percent higher than the IgM or IgG prior to administration. In still other embodiments, the IgM or IgG response to the polymer after a second or repeated intravenous or intramuscular administration of a pharmaceutical composition including an effective amount of PEOZ-lipid LNP of the present disclosure is less than 1 percent higher than the IgM or IgG prior to administration. In yet other embodiments, the IgM or IgG response to the polymer after a second or repeated intravenous or intramuscular administration of a pharmaceutical composition including an effective amount of PEOZ-lipid LNP of the present disclosure is less than 0.1 percent higher than the IgM or IgG prior to administration.

In addition, after a second administration of a PEOZ-lipid LNP of the present disclosure, the LNP is present in the blood or a tissue of the subject at a concentration of at least 75%, such as 80%, 85%, 90%, 95%, or greater, as compared to the first administration. In other aspects, a PEOZ-lipid LNP of the present disclosure has a reduced accelerated blood clearance after a second dose.

### EXAMPLES

The following non-limiting examples are merely illustrative of the preferred embodiments of the present invention, and are not to be construed as limiting the invention, the scope of which is defined by the appended claims.

### Materials

Triethylamine (TEA) was purchased from Sigma-Aldrich. Tetrahydrofuran (THF), dichloromethane (DCM), and acetonitrile (ACN) were purchased from EMD Millipore.

### Example 1. Synthesis of Compound 15b

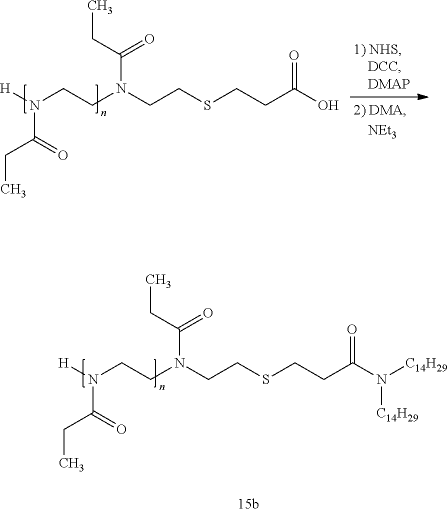

An oven-dried 250-mL round bottomed flask was charged with PEOZ-COOH (7.00 grams, 3.18 mmol, 1.00 equiv) followed by DCM (50 mL), N-hydroxysuccinimide (0.48 grams, 4.14 mmol, 1.30 equiv), and lastly DMAP (0.04 grams, 0.31 mmol, 0.1 equiv) under an atmosphere of Argon. DCC (0.854 g, 4.14 mmol, 1.30 equiv) was added in one portion, and the resulting solution was allowed to stir for at least 12 hours at room temperature. Following this time period, the reaction mixture was filtered through a coarse sintered glass frit, followed by rinsing the frit with additional DCM. The resulting solution was then slowly transferred to a beaker containing a stirred solution of Et2O (2000 mL). The precipitate was collecting via vacuum filtration, and the solids were dried under vacuum. The solids were then taken up into DCM (50 mL) in a dry 250 mL round bottomed flask equipped with a stir bar under an atmosphere of Argon. The reaction mixture was then charged with dimyristylamine (N,N-dimethyltetradecylamine or DMA) (2.6 g, 6.36 mmol, 2.00 equiv) followed by NEt₃ (0.89 mL, 6.36 mmol, 2.00 equiv), and the reaction mixture was allowed to stir for at least 12 hours at room temperature. After this time had passed, the reaction mixture was precipitated into a beaker containing a stirred solution of 2000 mL hexanes. The solids were collected via vacuum filtration and dried under vacuum. The solids were then dissolved in 200 mL deionized water and passed through an amberlite column containing 200 grams Amberlite IR-67 and 200 grams Amberlite IR-120H. The resulting water solution was concentrated to dryness on a rotary evaporator. The residue was taken up into dichloromethane (100 mL), dried with sodium sulfate and concentrated to afford 4.5 grams of the title compound.

¹H NMR analysis showed the standard backbone signals for PEOZ (500 MHz, CDCl₃) δ 3.64 (CH₂CH₂ backbone); 3.27 (N—CH₂); 2.72 (S—CH₂); 2.32-2.19 (C(O)—CH₂); 1.12 (CH₃). Additional signals were present for the lipid moiety at δ 1.49-1.54 (N—(CH₂)₂); 1.27 (CH₂); 0.87 (CH₃)

### Example 2. Synthesis of Compound 13

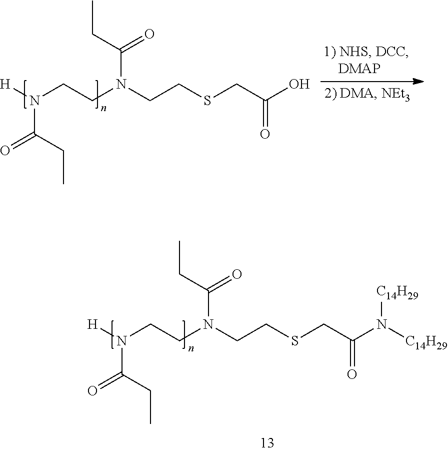

Compound 13 was prepared in an analogous fashion to compound 15B. 1.4 grams were isolated.

¹H NMR. 1H NMR analysis showed the standard backbone signals for PEOZ (500 MHz, CDCl₃) δ 3.43 (CH₂CH₂ backbone); 3.24 (N—CH₂); 2.8 (S—CH₂); 2.32-2.19 (C(O)—CH₂); 1.12 (CH₃). Additional signals were present for the lipid moiety at δ 1.49-1.65 (N—(CH₂)₂); 1.27 (CH₂); 0.87 (CH₃).

### Example 3. Synthesis of Compound 10

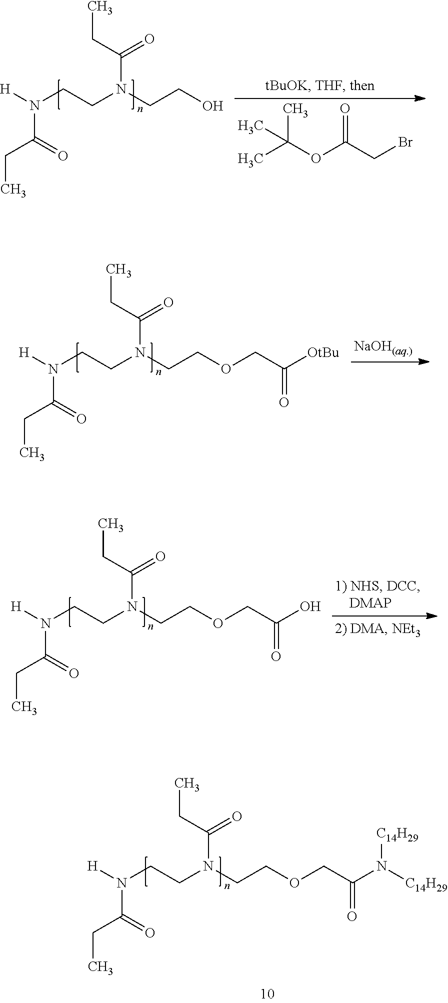

Poly(ethyloxazoline) (OH terminated, 0.5 grams, 0.27 mmol, 1.00 equiv) was dissolved in 30 mL THF and transferred to an oven-dried 250 mL round bottomed flask. Potassium tert-butoxide (0.09 g, 0.81 mmoL, 3.00 equiv) was added, and the reaction mixture was allowed to stir for 30 min at room temperature. Following this time period, tert-butyl bromoacetate (0.24 mL, 1.62 mmol, 6.00 equiv) was added, and the reaction mixture was allowed to stir at room temperature for at least 12 hours. Following this time period, the reaction mixture was transferred to a separatory funnel along with brine (50 mL). The mixture was extracted with CH₂Cl₂ (2×25 mL), and the combined organics were dried with sodium sulfate and concentrated in vacuo. The resulting solid was dissolved in a 1M aqueous solution of NaOH (20 mL) and stirred for at least 12 hours at room temperature. The solution was then acidified to pH=3, extracted with CH₂Cl₂ (2×30 mL), and the combined organics were dried with sodium sulfate and concentrated in vacuo to afford 0.4 grams of the intermediate carboxylic acid, which was used directly without further purification.

A 100 mL round bottomed flask was charged with the carboxylic acid from above (1 g, 0.45 mmol, 1.00 equiv) followed by CH₂Cl₂ (30 mL), DMAP (0.01 g), and lastly NHS (0.04 grams, 0.36 mmol, 2.00 equiv). DCC (0.08 g, 036 mmol, 2.00 equiv) was then added, and the reaction mixture was allowed to stir for at least 12 hours at room temperature. The resulting mixture was then filtered and concentrated to afford the intermediate NHS ester, which was used directly without further purification. The NHS ester was then transferred to a 100 mL round bottomed flask, and CH₂Cl₂ (30 mL) was added. Dimyristylamine (0.56 grams, 1.36 mmol, 3.00 equiv) was added followed by triethylamine (0.20 mL, 1.36 mmol, 3.00 equiv). The reaction mixture was stirred for at least 12 hours whereupon the mixture was diluted with brine (20 mL) and transferred to a separatory funnel. The mixture was extracted with CH2Cl2 (2×20 mL), and the combined organics were dried with sodium sulfate and concentrated in vacuo. The product was purified via reverse phase chromatography (C18, acetonitrile: methanol) to afford the 0.27 grams of the title compound.

¹H NMR analysis showed the standard backbone signals for PEOZ (500 MHz, DMSO) δ 4.27-3.93 (CH₂O and O—CH₂—N); 3.35 (CH₂CH₂ backbone); 2.96 (N—CH₂); 2.32-2.27 (C(O)—CH₂); 0.96 (CH₃). Additional signals were present for the lipid moiety at δ 1.49-1.54 (N—(CH₂)₂); 1.27 (CH₂); 0.87 (CH₃).

### Example 4. Preparation of PEG and PEOZ Lipid Nanoparticles containing DMA

Lipid stock solutions of ALC0315 (Cayman, 34337), 1,2-distearoyl-sn-glycero-3-phosphocholine [DSPC] (Bachem 4005619), Cholesterol ultrapure (VWR 0433) and 2 kDa polymer PEG conjugated DMA (Avanti, ALC-0159) or 2 kDa polymer PEOZ (as described in Example 1 above) were prepared in ethanol (EMD Millipore, EX0276-4). The lipid stock solutions were mixed at molar ratio of 46.3/9.4/42.7/1.6 of ALC0315/DSPC/cholesterol/PEG or PEOZ 2000 DMA in ethanol. The final lipid mixture stock (organic solution) contained 8.9 mg/mL of ALC-0315, 1.87 mg/mL of DSPC, 4.15 mg/mL of cholesterol and 1.0 mg/ml of polymer DMA corresponding to 15.95 mg/mL total lipid content in ethanol.

The plasmid DNA (pDNA) used in this example was the pWiz GFP plasmid DNA (Aldevron). The plasmid has 5757 bps and is supplied at 5.05 μg/μL in water and was diluted with 10 mM citrate buffer, pH 4, to prepare an aqueous solution of 0.2 mg/mL concentration.

A microfluidic mixing system (Nunchuck, Unchained Labs) equipped with a cartridge was used to mix the pDNA (aqueous) and the polymer lipid (organic) solution at a ratio of 3 parts aqueous to 1 part organic at a flow rate of 20 mL/min. The collected fractions for each PEG or PEOZ formulation were diluted with 10 mM Tris-HCl, pH 7 to reduce the ethanol from 25% to 6%. The size, polydispersity, and zeta potential of the formulated LNPs was measured in triplicate and averaged using dynamic light scattering (DLS) (Zetasizer Ultra, Malvern model ZSU3305) as shown in Table 1 below.

TABLE 1
Properties of Formulated LNPs

| | | | Zeta | |
|---|---|---|---|---|
| | Z-Average | Polydispersity | Potential | Conductivity |
| Sample Name | (nm) | Index (PI) | (mV) | (mS/cm) |
| POZ DMA LNP | 89.45 | 0.1353 | −7.1197 | 0.9654 |
| PEG DMA LNP | 92.14 | 0.1503 | −2.6229 | 0.9651 |

Amicon Ultra-4 Centrifugal Filter Units, 100 kDa MWCO, (Millipore, UFC810024) were used to complete buffer exchange into 10 mM Tris-HCl, pH 7 buffer and to concentrate formulations to final concentration. Solutions were filter sterilized with 13 mm 0.2 μm Acrodisc syringe filters with PES membrane (Pall, 4602).

Final formulations were assayed after concentration by assessing Particle Size, Polydispersity, Zeta, DNA concentration and % encapsulation using PicoGreen Assay, DNA Encapsulation by Gel Assay, and payload delivery by transfection of HEK293 Cells. Final Phospholipid concentration was calculated by using the initial LNP formulation ratio of DSPC to DNA (3.12) and multiplying by the measured DNA concentration in the final formulation.

Quant-iT PicoGreen dsDNA Assay Kit (P7589) was used to quantify “free non-encapsulated” and total pDNA in the formulations by analyzing the LNP formulations with and without the addition of a 2% Triton-X solution and quantified against a corresponding pDNA standard curve prepared from 1.0 to 0.02 μg/mL with and without addition of Triton-X. Plated standards and samples were measured at an excitation at 480 nm and emission at 520 nm on a Varioskan LUX plate reader. Percent (%) Encapsulation was quantified by:

$$\% Encapsulation = \frac{conc with TritonX ( total ) - conc witout TritonX ( free )}{conc with TritonX ( total )} * 100$$

Gel Assay Encapsulation was analyzed by loading about 100 ng of diluted pDNA GFP aqueous solution and diluted LNP formulations in Tris-HCl, pH 7.0 with and without addition of Triton-X onto a E-Gel EX with SYBR Gold II, 1% Agarose (Invitrogen, G401001) using the Invitrogen E-Gel Power snap system.

HEK293 cells were plated at 10,000 cells/well in cDMEM media (Corning, 10-013-CV) with 10% FBS in a 96 well treated plate (Costar, 3596) and allowed to grow overnight in incubator at 37° C./5% CO₂. Media was removed the following day, cells were washed with DPBS, and 50 μL of Opti-MEM media (Gibco, 11058-021) was replaced. 10, 20, and 50 μL of each formulation was added in triplicate to the plate and allowed to incubate for 4 hours. After 4 hours, 125 μL cDMEM media was added to the wells and allowed to incubate overnight. Transfected cells were viewed after 24 hours on EVOS M7000 (Invitrogen) microscope for GFP expression and all transfected cells were positive for GFP expression.

An 80% Sucrose (VWR, M117) solution was prepared in WFI water (Millipore, 4.96505.055) and filtered using a 0.2 μm PES sterile Vacuum Filtration Unit (VWR, 10040-436). The solution was added to the final formulations to bring the 2.0 mg/kg phospholipid formulations to 5% sucrose and the 0.2 mg/kg phospholipid formulations to 2% sucrose in order to adjust the osmolality of formulations within a range of 161-321 mOsm for injection into rats. The osmolality was measured by freeze point depression on a Fiske 210 micro osmometer instrument. Phospholipid concentrations were adjusted from calculated values by converting PicoGreen DNA concentrations to DSPC concentrations and then factoring in the dilution from adding sucrose. Table 2 below shows the size, polydispersity, zeta potential, and conductivity of the formulated LNPs. Table 3 shows the percent encapsulation.

TABLE 2
Properties of LNP after BXC

| | | | Zeta | |
|---|---|---|---|---|
| | Z-Average | Polydispersity | Potential | Conductivity |
| Sample Name | (nm) | Index (PI) | (mV) | (mS/cm) |
| PEG LNP | 88.26 | 0.1592 | −16.43 | 1.087 |
| POZ LNP | 106.3 | 0.2099 | −10.97 | 0.974 |

TABLE 3
Encapsulation

| | | | % En- | DNA | Calculated |
|---|---|---|---|---|---|
| | with | Without | capsu- | formula | DSPC based on |
| | TX | TX | lation | mg/mL | calculated DNA |
| PEG 0.2 LNP | 0.4075 | 0.01096 | 97.3 | 0.2038 | 0.6351 |
| POZ 0.2 LNP | 0.3959 | 0.01421 | 96.4 | 0.1980 | 0.6162 |
| PEG 2.0 LNP | — | — | — | 2.0375 | 0.6351 |
| POZ 2.0 LNP | — | — | — | 1.9795 | 0.6162 |

### Example 5. Quantification of Anti-PEG or Anti-PEOZ IgM to PEG-DMG and PEOZ-DMG

Applying the experimental methods from A. J. D. S. Sanchez, et al., Substituting Poly(ethylene glycol) Lipids with Poly(2-ethyl-2-oxazoline) Lipids Improves Lipid Nanoparticle Repeat Dosing. Adv. Healthcare Mater. 13, 2304033 (2024) where the Moderna LNP with aVHH mRNA using PEG-Dimyristoyl-rac-glycerol (DMG) or PEOZ-DMG was intramuscularly injected each hind leg of a C57BL/6J mouse with 3 μg mRNA, the inventors were able to conclude that both PEG-DMG and PEOZ-DMG form detectable IgM antibodies, but PEOZ-DMG forms less detectable IgM antibodies than PEG-DMG.

### Experimental Method

One hundred microliters carboxy-modified latex beads (Life Technologies, C37259) were coupled with 1-ethyl-3-(3-dimethylaminopropyl) carbodiimide HCl (5 mg) (TCI Chemicals, D1601-5G) for 15 min at room temperature (RT) on a plate shaker at maximum rpm. The activated beads were incubated with PEG-DMG 2k (2000 g/mol) or PEOZ-DMG 2k (12 μg) for 2 h. Beads were washed 2 times with PBS, resuspended in 2% MSD Blocker A (Meso Scale Diagnostics, R93AA), and stored at 4° C. overnight. Mouse serum samples (10 μL), isolated 48 h after administration of LNPs, were used at 1:100 dilutions and incubated with the beads at RT on a plate shaker for 1 h. Beads were incubated with Mouse IgM monoclonal (primary) antibody (Thermo Fisher Scientific, 14-5790-82) in the dark. Samples were washed and incubated with Goat anti-Rat IgG (H+L) Highly Cross-Adsorbed secondary antibody, Alexa Fluor Plus 647 (Thermo Fisher Scientific, A48265) for fluorescent detection. After further washing, beads were analyzed by flow cytometry.

Mice that received four injections of PEG-LNPs generated anti-PEG IgM. Similarly, mice that received four injections of PEOZ-LNPs generated anti-PEOZ IgM. However, the antibodies generated against PEOZ relative to PEG were reduced.

### Example 6. Quantification of Anti-PEG or Anti-PEOZ IgM or IgG Response to PEG-DMA and PEOZ-DMA

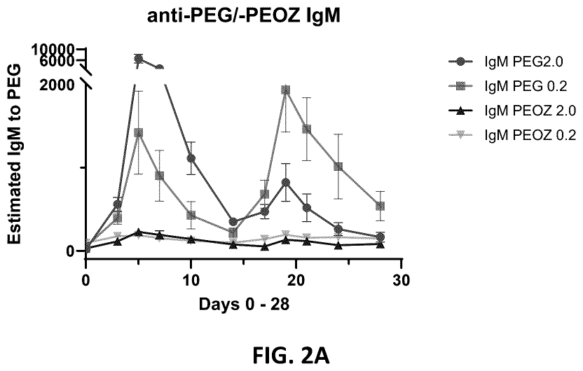

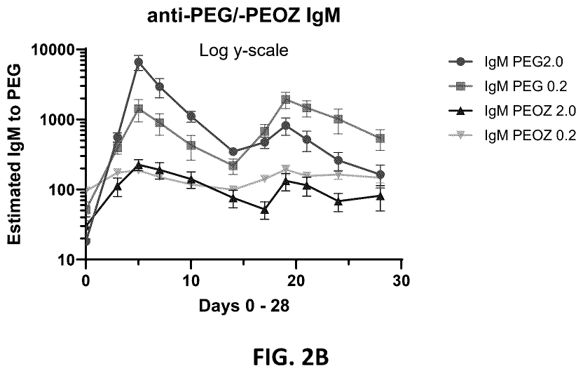

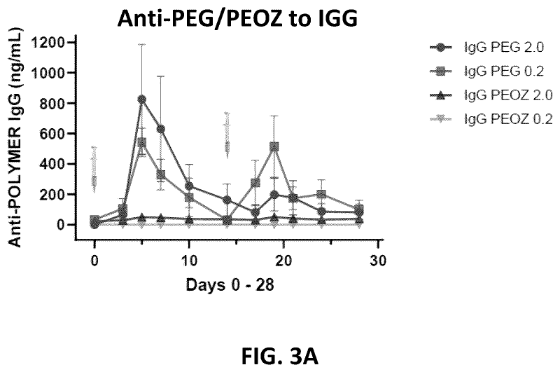

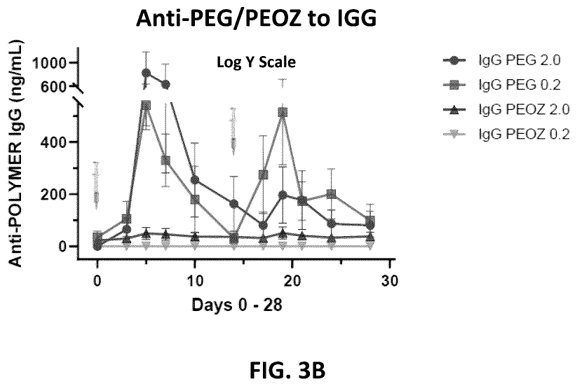

Using the PEG and PEOZ Lipid Nanoparticles containing DMA of Example 4, anti-PEG or anti-PEOZ IgM and IgG in rat plasma were detected by ELISA. Unlike PEG-DMA LNP which have a boosted anti-PEG IgM response after injections at day 0 and day 14, the PEOZ-lipid LNPs of the present disclosure do not induce a boosted anti-PEOZ IgM or IgG response after injections at day 0 and day 14. More specifically, as shown in FIGS. 2A and 2B for the IgM response, and FIGS. 3A and 3B for the IgG response, while both concentrations of the PEG-DMA LNP have a significant increase in IgM or Ig after the first and second injections (as expected), neither concentration of PEOZ-DMA LNP evaluated in this example raises the anti-PEOZ response by more than about 0.1 percent compared to the baseline initial IgM or IgG prior to administration.

### Experimental Method

Each well of a MaxiSorp 96-well microplate (Biolegend, 423501) was coated with 40 μg/well of PEG2K-CM (Layson Bio) or PEOZ 2K for IgM assay or 80 μg/well of PEG2K-CM or PEOZ 2K for IgG in 100 μL PBS (VWR, K812) overnight at 4° C. The standard curve lanes (1 and 2) were coated with PEG 2K. The remainder of the plate wells depended on the rat dose group being analyzed (PEG for the PEG LNPs dosed rats and PEOZ for the PEOZ LNPs dosed rats). After overnight incubation, the coated plate was washed three times with wash buffer (0.05% (w/v) CHAPS (VWR, 0465) in PBS. 200 μL of blocking buffer (5% (w/v) skim milk powder in PBS) was then added followed by incubation at room temperature for 1.5 hours.

Plates were then washed three times wash buffer. Dilutions of rat anti-PEG IgM (rAGP6) and of rat anti-PEG IgG (r33G) were prepared in dilution buffer (2% (w/v) skim milk powder in PBS) for a standard curve. (1000, 333.3, 111.1, 37, 12.4, 4.1, and 1.4 ng/ml for IgG and 200, 80, 32, 12.8, 5.12, 2.048, and 0.8192 ng/mL for IgM). Rat plasma samples were diluted 1:100 for IgM and 1:50 for IgG analysis with dilution buffer. 100 μL of the Standards and Samples was plated onto the 96 well plate and then incubated for 1 hour at room temperature. Plates were subsequently washed five times with wash buffer to remove unbound antibodies. 50 μL of 0.08 μg/mL of peroxidase-conjugated AffiniPure™ Rabbit anti-rat IgM (Jackson Immuno Research, 312-035-020) or Peroxidase-conjugated AffiniPure™ donkey anti-rat IgG (H+L) (Jackson Immuno Research, 712-035-150) in dilution buffer was added to the IgM or IgG detection plates, respectively, and then incubated for 1 hour at room temperature. The plates were subsequently washed five times with wash buffer. 100 μL of TMB solution (Thermo Scientific, 34021) was added to plates and incubated for 20-30 minutes at room temperature to allow color development.

The HRP reaction was stopped with the addition of 100 μL of 2 NH₂SO₄ (Macron, H381-05). The absorbance was measured at 450 nm on a Varioskan LUX microplate reader and the 450 nm blank dilution buffer signal was subtracted to calculate for a corrected absorbance. The concentrations of anti-PEG IgG and anti-PEG IgM in serum samples were calculated using four-parameter logistic (4PL) curves for anti-PEG IgM (rAGP6) and anti-PEG IgG (r33G) fitted by SkanIt 7.0.2 software.

This is a surprising and unexpected result considering that LNPs including PEG-1,2-Dimyristoyl-rac-glycerol (DMG) 2k and LNPs including PEOZ-DMG 2k and a payload of mRNA encoding luciferase (Example 7) both elicit an IgM response. More specifically, while PEOZ-DMG 2k elicits less of an IgM response (as compared to its PEG-DMG 2k counterpart), it does still cause detectable antibodies to be generated against PEOZ-DMG (A. J. D. S. Sanchez et al., Substituting Poly (ethylene glycol) Lipids with Poly(2-ethyl-2-oxazoline) Lipids Improves Lipid Nanoparticle Repeat Dosing. Adv. Healthcare Mater. 13, 2304033 (2024)).

The PEOZ-lipid LNPs and pharmaceutical compositions described and claimed herein are not to be limited in scope by the specific embodiments herein disclosed, since these embodiments are intended as illustrations of several aspects of the disclosure. Any equivalent embodiments are intended to be within the scope of this disclosure. Indeed, various modifications of the formulas and structures in addition to those shown and described herein will become apparent to those skilled in the art from the foregoing description. Such modifications are also intended to fall within the scope of the appended claims. All patents and patent applications cited in the foregoing text are expressly incorporated herein by reference in their entirety. Any section headings herein are provided only for consistency with the suggestions of 37 C.F.R. § 1.77 or otherwise to provide organizational queues. These headings shall not limit or characterize the invention(s) set forth herein.

What is claimed:

1. A method of delivering a payload in a subject without raising a PEOZ-associated immune response comprising:
providing a lipid nanoparticle composition comprising:
a compound of Formula I 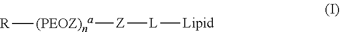
wherein R comprises an initiating group, PEOZ comprises a polymer of the structure [N(COR₂)CH₂CH₂], wherein R₂ is ethyl, n ranges from 1 to 1,000, a is ran, which indicates a random copolymer, or block, which indicates a block copolymer, Z comprises S, O, or N, L comprises a linking group with controllable degradability in physiological media, and Lipid comprises dimyristylamine;
an ionizable or cationic lipid or cationic POZ;
a helper lipid;
a sterol lipid; and
a payload; and
administering to the subject an effective amount of the lipid nanoparticle composition, wherein the subject has a first amount of anti-PEOZ IgG and/or anti-PEOZ IgM prior to administration and a second amount of anti-PEOZ IgG and/or anti-PEOZ IgM after administration.

2. The method of claim 1, wherein the PEOZ has a molecular weight between 500 Daltons and 5,000 Daltons.

3. The method of claim 2, wherein the PEOZ has a molecular weight between 1,500 Daltons and 3,000 Daltons.

4. The method of claim 1, wherein R comprises a hydrogen or a substituted or unsubstituted alkyl.

5. The method of claim 1, wherein Formula I is: 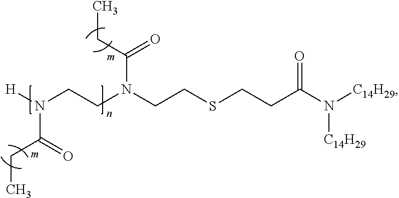
wherein m is 1, n ranges from 1 to 1000, and p ranges from 1 to 10.

6. The method of claim 1, wherein L is —CH₂CH₂—G—, and wherein G comprises the linking group.

7. The method of claim 6, wherein G comprises ethers, esters, carboxylate esters, carbonate esters, carbamates, amines, amides, disulfides, or combinations thereof.

8. The method of claim 1, wherein R comprises a hydrogen, or a substituted or unsubstituted alkyl, and wherein n ranges from 15 to 35.

9. The method of claim 1, wherein the step of administering comprises delivering the pharmaceutical composition to the animal via subcutaneous, intravenous, intramuscular, intradermal or aerosol routes.

10. The method of claim 1, wherein the second amount is less than 2 percent higher than the first amount.

11. The method of claim 10, wherein the second amount is less than 1 percent higher than the first amount.

12. The method of claim 11, wherein the second amount is less than 0.1 percent higher than the first amount.

13. A method for raising a protective immune response in an animal without raising a PEOZ-associated immune response, comprising the steps of:
providing a lipid nanoparticle composition comprising
a compound of Formula I 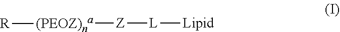
wherein R comprises an initiating group, PEOZ comprises a polymer of the structure [N(COR₂)CH₂CH₂], wherein R₂ is ethyl, n ranges from 1 to 1,000, a is ran, which indicates a random copolymer, or block, which indicates a block copolymer, Z comprises S, L comprises —CH₂CH₂—G—, and Lipid comprises dimyristylamine;
an ionizable lipid;
a helper lipid;
a sterol lipid; and
an oligonucleotide payload; and
administering to the animal an effective amount of the lipid nanoparticle composition, wherein the animal has a first amount of anti-PEOZ IgM and/or anti-PEOZ IgG antibodies prior to administration and a second amount of anti-PEOZ IgM and/or anti-PEOZ IgG antibodies after administration.

14. The method of claim 13, further comprising repeating the step of administering after a predetermined amount of time.

15. The method of claim 13, wherein G comprises a linking group.

16. The method of claim 13, wherein the PEOZ has a molecular weight between 500 Daltons and 5,000 Daltons.

17. The method of claim 15, wherein the PEOZ has a molecular weight between 1,500 Daltons and 3,000 Daltons.

18. The method of claim 13, wherein R comprises a hydrogen or a substituted or unsubstituted alkyl.

19. The method of claim 13, wherein Formula I is: 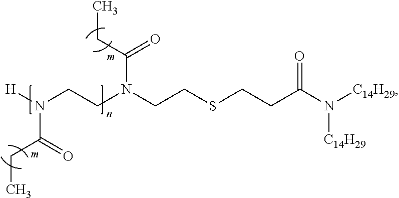
wherein m is 1, n ranges from 1 to 1000, and p ranges from 1 to 10.

20. The method of claim 13, wherein G comprises ethers, esters, carboxylate esters, carbonate esters, carbamates, amines, amides, disulfides, or combinations thereof.

21. The method of claim 13, wherein R comprises a hydrogen, or a substituted or unsubstituted alkyl, and wherein n ranges from 15 to 35.

22. The method of claim 13, wherein the step of administering comprises delivering the pharmaceutical composition to the animal via subcutaneous, intravenous, intramuscular, intradermal or aerosol routes.

23. The method of claim 1, wherein the second amount is less than 5 percent higher than the first amount.

24. The method of claim 11, wherein the second amount is less than 2 percent higher than the first amount.

25. The method of claim 11, wherein the second amount is less than 0.1 percent higher than the first amount.

26. A method for raising a protective immune response in a subject having a first amount of anti-PEOZ IgG and/or anti-PEOZ IgM comprising:
providing a lipid nanoparticle composition comprising:
a compound of Formula I 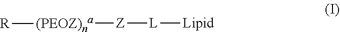
wherein R comprises an initiating group, PEOZ comprises a polymer of the structure [N(COR₂)CH₂CH₂], wherein R₂ is ethyl, n ranges from 1 to 1,000, a is ran, which indicates a random copolymer, or block, which indicates a block copolymer, Z comprises S, O, or N, L comprises a linking group with controllable degradability in physiological media, and Lipid comprises dimyristylamine;
an ionizable or cationic lipid or cationic POZ;
a helper lipid;
a sterol lipid; and
a payload; and
administering to the subject an effective amount of the lipid nanoparticle composition, wherein the subject has a second amount of anti-PEOZ IgG and/or anti-PEOZ IgM after administration that is less 10 percent higher than the first amount.

27. The method of claim 26, wherein the second amount is less than 5 percent higher than the first amount.

28. The method of claim 27, wherein the second amount is less than 2 percent higher than the first amount.

29. The method of claim 28, wherein the second amount is less than 0.1 percent higher than the first amount.

30. The method of claim 26, wherein the PEOZ has a molecular weight between 1,500 Daltons and 3,000 Daltons.
